(19) United States
(12) Patent Application Publication
(10) Pub. No.: US 2024/0315631 A1
(43) Pub. Date: Sep. 26, 2024

(54) EVALUATION METHOD, EVALUATION DEVICE, AND COMPUTER PROGRAM FOR EVALUATING NEUROTRANSMITTER SIGNALS CONDUCTED THROUGH AXON BUNDLES

(71) Applicant: SHARP KABUSHIKI KAISHA, Sakai City, Osaka (JP)

(72) Inventors: Yuko HIRABE, Sakai City, Osaka (JP); SHIGENORI KINOSHITA, Sakai City, Osaka (JP); Yasunari HOSOI, Sakai City, Osaka (JP); TOMOKO TERANISHI, Sakai City, Osaka (JP); Satoshi IHIDA, Sakai City, Osaka (JP)

(21) Appl. No.: 18/577,164

(22) PCT Filed: Jul. 9, 2021
(86) PCT No.: PCT/JP2021/026015

§ 371 Date: Jan. 5, 2024

(51) Int. Cl. A61B 5/294

(52) U.S. Cl. CPC A61B 5/294

(57) ABSTRACT

An evaluation method for evaluating a neurotransmission signal that is conducted through an axon bundle includes: performing a leveling that estimates a baseline of a signal waveform and subtracts an estimated baseline from the signal waveform; grouping a plurality of peaks that is contained in the signal waveform after the leveling into a plurality of groups each corresponding to one of the axons included in the axon bundle; calculating a similarity between a first signal waveform acquired by a first measurement electrode among a plurality of measurement electrodes and a second signal waveform acquired by a second measurement electrode, which is distinct from the first measurement electrode, after the grouping; and calculating a signal conduction time between the first measurement electrode and the second measurement electrode, based on a calculated similarity.

### TECHNICAL FIELD

The present disclosure relates to an evaluation method, an evaluation apparatus, and a computer program for evaluating a neurotransmission signal that is conducted through an axon bundle.

### BACKGROUND ART

Neurodegenerative diseases are known as incurable diseases of the modern era. Neurodegenerative diseases are diseases in which neurodegeneration occurs for unknown causes. An example of a neurodegenerative disease is amyotrophic lateral sclerosis (ALS). In ALS, neurodegeneration causes muscular atrophy, whereby the body gradually becomes immobile. Neurodegeneration means that degenerative changes occur in nerve cells. A nerve cell is composed of a cell body, an axon, and dendrites.

One reason for the current lack of sufficient progress in identifying the causes of neurodegenerative diseases and in drug discovery research may be the fact that nerve cells that fully reproduce the patient's pathology are not available in large quantities, because it is impossible to collect large quantities of nerve cells from an actual patient.

Therefore, organoids (artificial nerves) that have differentiated from iPS cells (induced Pluripotent Stem cells), which can reproduce patients' pathology, are drawing attention for their availability in research. One known organoid is a 2D organoid, which is obtained by seeding cell bodies in an MEA (Micro Electrode Array) having an array of measurement electrodes, and culturing them to allow axons to randomly extend from the cell bodies. Nerve development and functionality as well as pathological progress (e.g., axonal degeneration (axonal regression)) and degree, which are important factors in promoting 2D organoid research, are recognized through neurotransmission signal measurement. “Neurotransmission signal measurement” is, regarding two specific points, a measurement of similarity between a signal that is acquired at one point and a signal that is acquired at the other point. In a known neurotransmission signal measurement, by taking advantage of the tendency that axons randomly extend from a cell body that is seeded on an MEA and connect to other cell bodies, means are taken to prevent axons from overlapping and tangling, so that a single axon can be subjected to measurement.

On the other hand, in recent years, culture and growth of 3D organoids utilizing a microfluidic device (disclosed in e.g. Patent Document 1) have begun to attract attention. A 3D organoid is more similar in structure to a nerve cell within the human body than a 2D organoid. In a nerve cell within the human body, axons extending form a mass of cell bodies belonging to multiple nerve cells spontaneously become entangled to form a bundle (called an “axon bundle”). Currently, research is actively under way for drug discovery screening and etiological studies utilizing 3D organoids.

### CITATION LIST

### Patent Literature

[Patent Document 1] Japanese Laid-Open Patent Publication No. 2020-151784

### Non-Patent Literature

[Non-Patent Document 1] P. H. C. Eilers and H. F. M. Boelens, “Baseline correction with asymmetric least squares smoothing,” vol. 1, no. 1, Leiden University Medical Centre, Leiden, Netherlands, 2005, Leiden University Medical Centre Report.

### SUMMARY

### Technical Problem

Known methods of measuring neurotransmission signals cannot be applied to a structure that has an axon bundle, such as a 3D organoid. In other words, no method for evaluating a neurotransmission signal that is conducted through an axon bundle has been established.

Embodiments of the present disclosure have been made in view of the above problems, and an objective thereof is to provide an evaluation method and an evaluation apparatus which can evaluate a neurotransmission signal that is conducted through an axon bundle.

### Solution to Problem

According to embodiments of the present disclosure, solutions as recited in the following Items are provided.

### [Item 1]

An evaluation method for evaluating a neurotransmission signal that is conducted through an axon bundle which is a bundle of axons of a plurality of nerve cells, the evaluation being based on a signal waveform that is acquired by using each of a plurality of measurement electrodes disposed at respectively different positions relative to the axon bundle, the evaluation method comprising:

a leveling step of performing a leveling that estimates a baseline of the signal waveform and subtracts the estimated baseline from the signal waveform;

a grouping step of grouping a plurality of peaks that are contained in the signal waveform after having undergone the leveling into a plurality of groups each corresponding to one of the axons included in the axon bundle;

a similarity calculation step of calculating a similarity between a first signal waveform acquired by a first measurement electrode among the plurality of measurement electrodes and a second signal waveform acquired by a second measurement electrode which is distinct from the first measurement electrode after the grouping step; and

a conduction time calculation step of calculating a signal conduction time between the first measurement electrode and the second measurement electrode based on the calculated similarity.

### [Item 2]

The evaluation method of Item 1, wherein, in the leveling step, the baseline is estimated by using an asymmetric least squares method.

### [Item 3]

The evaluation method of Item 1 or 2, wherein, in the grouping step, with respect to a given pair of peaks among the plurality of peaks, a time difference between the peaks is calculated, and grouping is performed based on the calculated time difference and peak heights of the pair of peaks.

### [Item 4]

The evaluation method of any of Items 1 to 3, wherein, in the similarity calculation step, for vicinities of all peaks in the first signal waveform that belong to a certain group, inner product values with a second signal waveform obtained by being shifted by a predetermined shift amount in a time axis direction are calculated and added up, a resultant value thereof being determined as the similarity.

### [Item 5]

The evaluation method of Item 4, wherein,

the similarity calculation step is performed a plurality of times while varying the shift amount; and,

in the conduction time calculation step, a shift amount that maximizes the similarity is determined as the signal conduction time.

### [Item 6]

An evaluation apparatus for evaluating a neurotransmission signal that is conducted through an axon bundle which is a bundle of axons of a plurality of nerve cells, the evaluation being based on a signal waveform that is acquired by using each of a plurality of measurement electrodes disposed at respectively different positions relative to the axon bundle, the evaluation apparatus comprising:

a leveling section to perform a leveling that estimates a baseline of the signal waveform and subtracts the estimated baseline from the signal waveform;

a grouping section to group a plurality of peaks that are contained in the signal waveform after having undergone the leveling into a plurality of groups each corresponding to one of the axons included in the axon bundle;

a similarity calculation section to calculate a similarity between a first signal waveform acquired by a first measurement electrode among the plurality of measurement electrodes and a second signal waveform acquired by a second measurement electrode which is distinct from the first measurement electrode after the grouping by the grouping section; and

a conduction time calculation section to calculate a signal conduction time between the first measurement electrode and the second measurement electrode based on the calculated similarity.

### [Item 7]

The evaluation apparatus of Item 6, wherein the leveling section estimates the baseline by using an asymmetric least squares method.

### [Item 8]

The evaluation apparatus of Item 6 or 7, wherein, with respect to a given pair of peaks among the plurality of peaks, the grouping section calculates a time difference between the peaks, and performs grouping based on the calculated time difference and peak heights of the pair of peaks.

### [Item 9]

The evaluation apparatus of any of Items 6 to 8, wherein, for vicinities of all peaks in the first signal waveform that belong to a certain group, the similarity calculation section calculates and adds up inner product values with a second signal waveform obtained by being shifted by a predetermined shift amount in a time axis direction, a resultant value thereof being determined as the similarity.

### [Item 10]

The evaluation apparatus of Item 9, wherein,

the similarity calculation section calculates the similarity a plurality of times while varying the shift amount; and

the conduction time calculation section determines a shift amount that maximizes the similarity as the signal conduction time.

### [Item 11]

A computer program for evaluating a neurotransmission signal that is conducted through an axon bundle which is a bundle of axons of a plurality of nerve cells, the evaluation being based on a signal waveform that is acquired by using each of a plurality of measurement electrodes disposed at respectively different positions relative to the axon bundle, the computer program causing a computer to execute:

a leveling step of performing a leveling that estimates a baseline of the signal waveform and subtracts the estimated baseline from the signal waveform;

a grouping step of grouping a plurality of peaks that are contained in the signal waveform after having undergone the leveling into a plurality of groups each corresponding to one of the axons included in the axon bundle;

a similarity calculation step of calculating a similarity between a first signal waveform acquired by a first measurement electrode among the plurality of measurement electrodes and a second signal waveform acquired by a second measurement electrode which is distinct from the first measurement electrode after the grouping step; and

a conduction time calculation step of calculating a signal conduction time between the first measurement electrode and the second measurement electrode based on the calculated similarity.

### Advantageous Effects of Invention

According to embodiments of the present disclosure, an evaluation method and an evaluation apparatus which can evaluate a neurotransmission signal that is conducted through an axon bundle are provided.

### BRIEF DESCRIPTION OF DRAWINGS

FIG. 1 A diagram schematically showing a nerve cell 1.

FIG. 2 A diagram schematically showing a 3D organoid 5.

FIG. 3A A plan view schematically showing an incubator (microfluidic device) 10 as an example means with which the 3D organoid 5 can be cultured.

FIG. 3B A cross-sectional view schematically showing the incubator 10.

FIG. 3C A diagram showing cell bodies 2 being seeded in a first chamber 11a of the incubator 10.

FIG. 3D A diagram showing axons 3 extending into a channel 11c from the cell bodies 2 within the first chamber 11a.

FIG. 4A A plan view showing another example means with which the 3D organoid 5 can be cultured.

FIG. 4B A plan view showing the other example means with which the 3D organoid 5 can be cultured.

FIG. 5A A plan view showing still another example means with which the 3D organoid 5 can be cultured.

FIG. 5B A plan view showing the still other example means with which the 3D organoid 5 can be cultured.

FIG. 5C A plan view showing the still other example means with which the 3D organoid 5 can be cultured.

FIG. 6 (a) to (e) are diagrams for describing reasons why a composite signal is generated in the 3D organoid 5.

FIG. 7 (a) to (c) are diagrams for describing reasons why a measurement signal is considerably affected by changes in potential that are commensurate the distance between a measurement electrode 13 and an axon 3.

FIG. 8 A graph showing an example of a measurement signal suffering from a baseline drift.

FIG. 9 A diagram showing an overall configuration of the measurement system 200.

FIG. 10 A block diagram showing an example hardware configuration of an evaluation apparatus 100 according to an embodiment of the present disclosure.

FIG. 11 A functional block diagram showing the evaluation apparatus 100 in functional blocks.

FIG. 12 A flowchart showing an example processing procedure (algorithm) by the evaluation apparatus 100.

FIG. 13 A graph showing an example of subtracting an estimated baseline from an actual signal waveform.

FIG. 14A A graph showing eight peaks A to H that are subject to grouping.

FIG. 14B A graph where a given pair among eight peaks is denoted in the order of the earlier peak and the later peak, and each pair is plotted with the time difference between the peaks reading on the horizontal axis and the peak height of the later peak reading on the vertical axis.

FIG. 15 A flowchart of a specific technique of similarity calculation and conduction time calculation.

FIG. 16 A diagram showing an example of peak vicinities that may be extracted from a first signal waveform SW1.

FIG. 17 A diagram showing an example of extraction intervals of a second signal waveform SW2.

FIG. 18 A diagram showing a state where the second signal waveform SW2 is shifted by a predetermined shift amount Δt in a time axis direction.

FIG. 19 A graph to visualize a relationship between the shift amount Δt and the similarity where the shift amount Δt reads on the horizontal axis and the similarity reads on the vertical axis.

FIG. 20 A diagram showing a state where the second signal waveform SW2 has been shifted by the shift amount Δti that maximizes the similarity.

FIG. 21 A graph to visualize a relationship between the shift amount Δt and the similarity where the shift amount Δt reads on the horizontal axis and the similarity reads on the vertical axis.

FIG. 22 A flowchart of a specific technique of similarity calculation and conduction time calculation.

### DESCRIPTION OF EMBODIMENTS

Hereinafter, with reference to the drawings, embodiments of the present disclosure will be described. Note that embodiments of the present disclosure are not limited to the configuration exemplified below.

As will be described below, an evaluation method and an evaluation apparatus according to embodiments of the present disclosure are able to suitably evaluate a neurotransmission signal that is conducted through an axon bundle, which is a bundle of axons of a multitude of nerve cells. Before describing the evaluation method and the evaluation apparatus, the structure of a nerve cell, the structure of a 3D organoid including multiple nerve cells, means for culturing a 3D organoid, and the like will be described.

### [Nerve Cell]

FIG. 1 is a diagram schematically showing a nerve cell 1. The nerve cell 1 has a function of, when a stimulus is input to the nerve cell 1, generating an action potential for propagating information to other cells. As shown in FIG. 1, the nerve cell 1 includes a cell body 2, an axon 3, and dendrites (not shown here). The axon 3 and the dendrites may collectively be referred to as neurites.

The cell body 2 is a place in the nerve cell 1 where organelles such as a nucleus 2a are concentrated. The cell body 2 has a diameter on the order of several μm to ten and several μm.

The axon 3 is a part in the nerve cell 1 that is responsible for outputting signals to other cells. The axon 3 is structured as a projection that extends from the cell body 2. Basically only one axon 3 extends from a single cell body 1, but it may branch out. The end of the axon 3 opposite to the cell body 2 (called an “axon terminal”) 3a is connected to other cells.

A dendrite receives signals from other cells (i.e., they are responsible for inputs). A dendrite is structured so as to spread while ramifying as if branches of a tree from the cell body 2. More than one dendrite may exist in a single nerve cell 1.

### [3D Organoid]

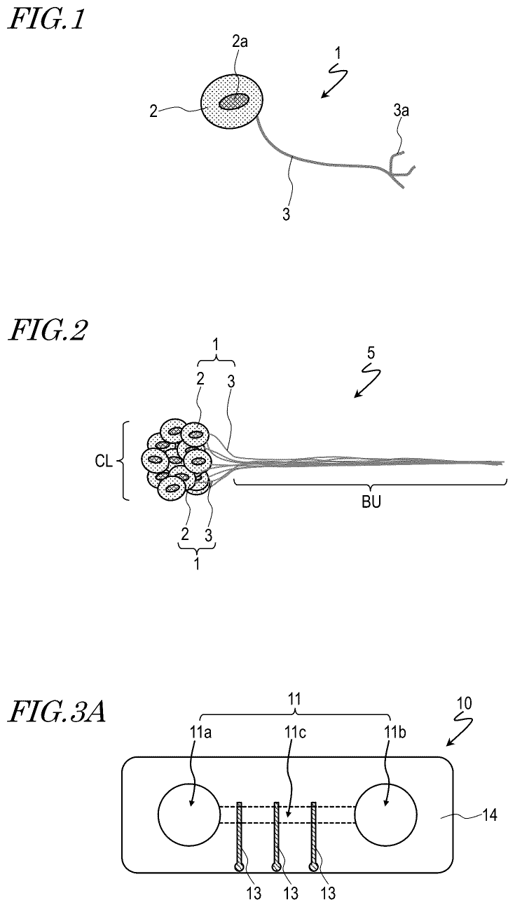

FIG. 2 is a diagram schematically showing a 3D organoid 5. As shown in FIG. 2, the 3D organoid 5 includes multiple nerve cells 1. The number of nerve cells 1 included in the 3D organoid 5 may be e.g. several thousand to ten and several thousand.

In the 3D organoid 5, the cell bodies 2 of multiple nerve cells 1 make a mass CL. Moreover, the axons 3 of the multiple nerve cells 1 make a bundle. In other words, the 3D organoid 5 is created (cultured) so that the axon bundle BU, being a bundle of axons 3, extends from the mass CL of cell bodies 2.

### [3D Organoid Culture Means]

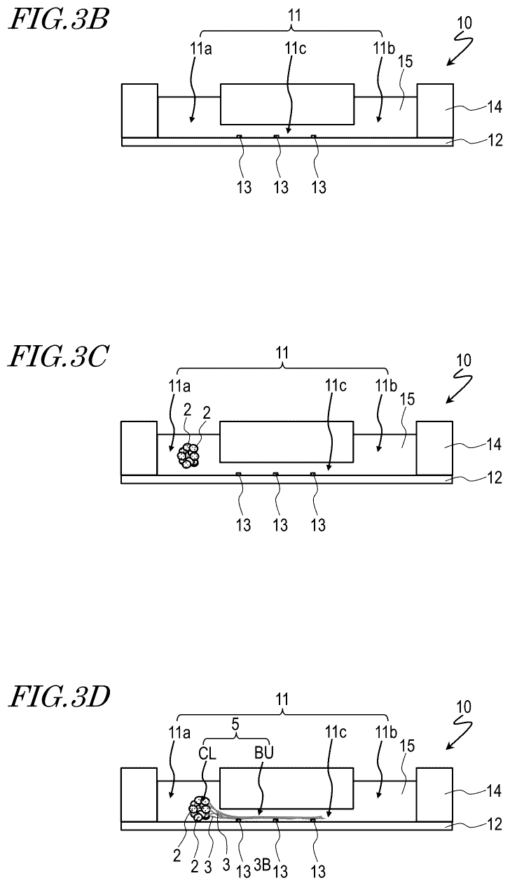

With reference to FIG. 3A and FIG. 3B, an incubator (microfluidic device) 10 as an example means with which the 3D organoid 5 can be cultured will be described. FIG. 3A and FIG. 3B are a plan view and a cross-sectional view schematically showing the incubator 10.

As shown in FIG. 3A and FIG. 3B, the incubator 10 includes an incubation space 11. The incubation space 11 is a space for culturing the 3D organoid 5.

In the illustrated example, the incubator 10 includes a substrate 12, and a plurality of measurement electrodes 13 and an upper plate 14 that are provided upon the substrate 12. The aforementioned incubation space 11 is created in the upper plate 14.

The substrate 12 is a plate member having a substantially rectangular shape in a plan view. The substrate 12 is a glass substrate, for example.

As will be described later, the plurality of measurement electrodes 13 are used for the measurement of neurotransmission signals. The measurement electrodes 13 are made of an electrically conductive material such as silver, copper, or aluminum.

The incubation space 11 includes a first chamber 11a and a second chamber 11b and a channel 11c. The channel 11c interconnects lower portions of the first chamber 11a and the second chamber 11b.

The plurality of measurement electrodes 13 are disposed so as to overlap the channel 11c in a plan view. The number of measurement electrodes 13 is not limited to what is illustrated in FIG. 3A and FIG. 3B.

In the incubation space 11 (i.e., in the first chamber 11a, the second chamber 11b, and the channel 11c), a culture fluid 15 is placed.

When the 3D organoid 5 is cultured in the incubator 10, first, as shown in FIG. 3C, multiple cell bodies 2 are seeded in the first chamber 11a in a closely huddled state. At this point, the axon 3 has not grown out of each cell body 2 yet. As the culture progresses, as shown in FIG. 3D, the axon 3 extends from each cell body 2, such that a number of axons 3 make a bundle within the channel 11c. In this manner, a 3D organoid 5 is created which is structured so that the axon bundle BU extends from the mass CL of cell bodies 2. The mass CL of cell bodies 2 is located in the first chamber 11a. When viewed from the normal direction of the substrate 12 (i.e., in a plan view), the axon bundle BU overlaps the plurality of measurement electrodes 13 within the channel 11c.

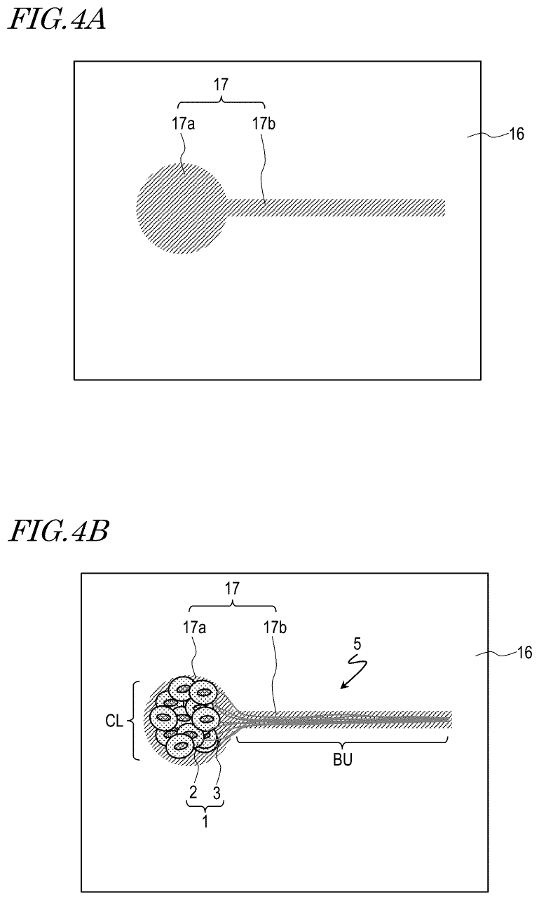

FIG. 4A shows another example means for culturing the 3D organoid 5. In the example shown in FIG. 4A, a cell-adhesive coating layer 17 is formed in a predetermined pattern on the cultivation substrate 16. The cultivation substrate 16 may be what is used for culturing 2D organoids. The coating layer 17 includes a first region 17a of a substantially circular shape and a second region 17b extending in a linear shape from the first region 17a.

When multiple cell bodies 2 are seeded on the first region 17a of the coating layer 17 in a closely huddled state, as shown in FIG. 4B, an axon 3 extends from each cell body 2 along the second region 17b of the coating layer 17, a number of axons 3 make a bundle within the second region 17b. In this manner, a 3D organoid 5 which is structured so that the axon bundle BU extends from the mass CL of cell bodies 2 can be created.

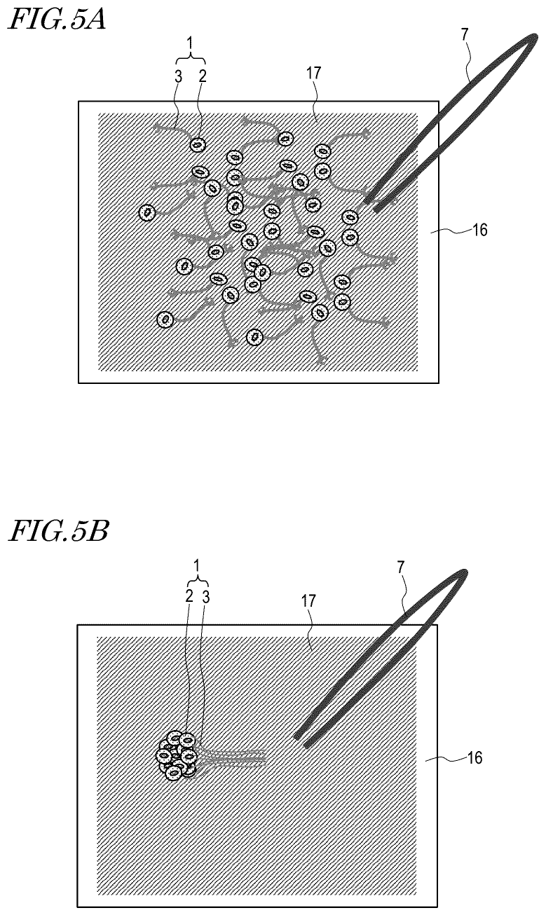

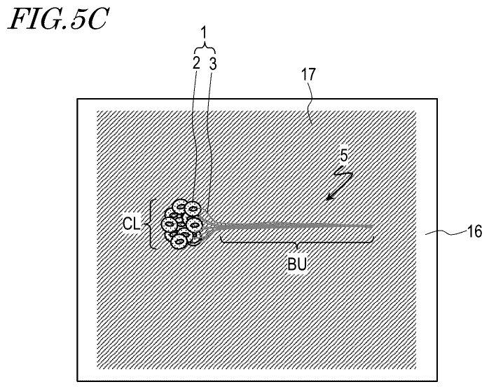

Alternatively, it is also possible to create a 3D organoid 5 in a manner shown in FIG. 5A, FIG. 5B and FIG. 5C. First, as shown in FIG. 5A, a cultivation substrate 16 having a cell-adhesive coating layer 17 formed on substantially the entire surface thereof is provided, and multiple cell bodies 2 are seeded on the coating layer 17. Then, the axons 3 which have grown from the cell bodies 2 are gathered by using tweezers 7 or the like to allow the axons 3 to turn into a bundle, as shown in FIG. 5B. In this manner, as shown in FIG. 5C, a 3D organoid 5 which is structured so that the axon bundle BU extends from the mass CL of cell bodies 2 can be created.

### Problems in Evaluation of a Neurotransmission Signal that is Conducted Through an Axon Bundle

Problems associated with evaluating a neurotransmission signal that is conducted through the axon bundle 5 will be described.
(1) As has already been described, the measurement electrodes 13 are provided on the microfluidic device (incubator) 10 with predetermined interspaces. However, each axon 3 in the axon bundle BU of the 3D organoid 5 may not necessarily be in direct contact with the measurement electrodes 13; moreover, the axon bundle BU has a structure in which axons 3 in projection shape have spontaneously become entangled to form a bundle. Therefore, the distance between a measurement electrode 13 and each axon 3 in the axon bundle BU cannot be controlled.

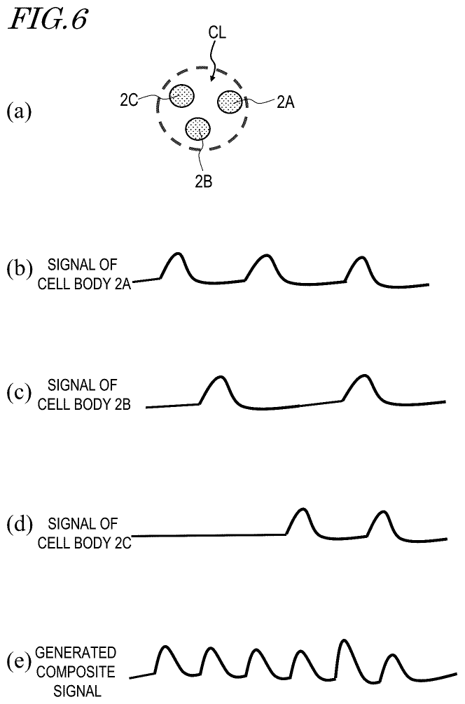

Moreover, the cell bodies 2 of the nerve cells 1 each emit a continuous subtle signal in a disparate manner. Furthermore, the speed with which a neurotransmission signal conducts (nerve conduction velocity) differs from axon 3 to axon 3 due to the degree of development (thickness and length) of the axon 3, and even the same axon 3 may have a different nerve conduction velocity from day to day. Therefore, a signal that is measured by each measurement electrode 13 is a composite signal that is generated as a result of the disparately-emitted continuous signals from the multiple cell bodies 2 having been conducted at a different nerve conduction velocity for each axon 3. For example, when signals as shown in FIGS. 6(b), (c) and (d) are disparately emitted from three cell bodies 2A, 2B and 2C as shown in FIG. 6(a), a composite signal as shown in FIG. 6(e) is emitted.
(2) In order to capture subtle signals, the microfluidic device 10 measures a potential within a cell as a capacitance, where the cell membrane of any axon 3 above a measurement electrode 13 is regarded as a dielectric of a capacitor, thereby obtaining a measurement signal (signal waveform).

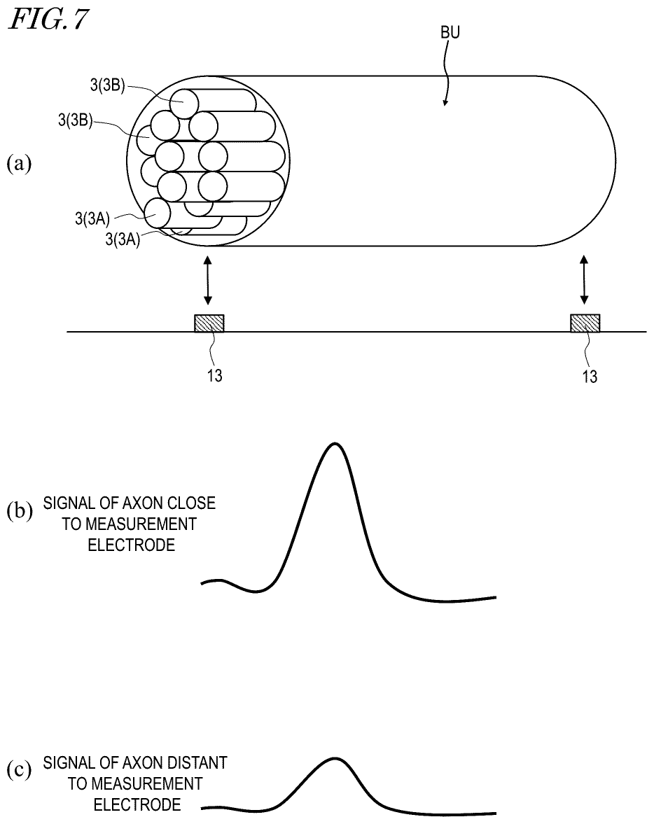

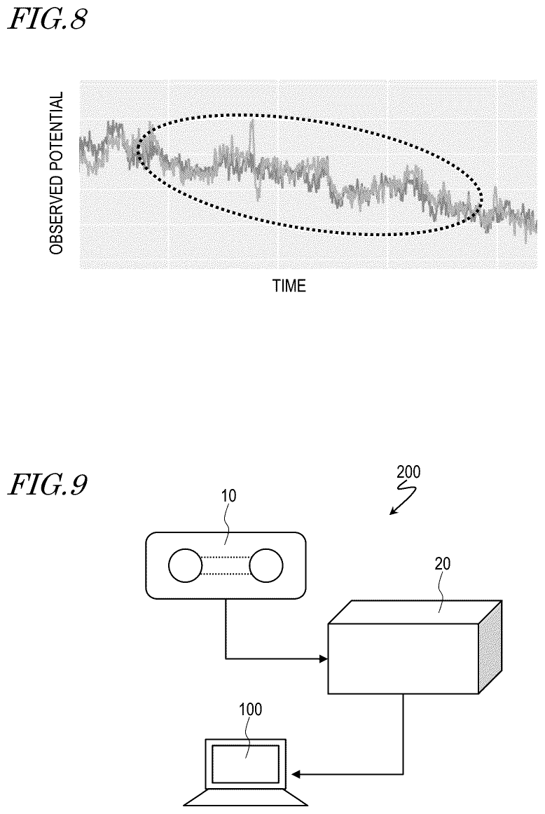

On the other hand, the magnitude of the potential of a measurement signal that is obtained from each axon 3 in the axon bundle BU that is located above a measurement electrode 13 depends on the distance between the measurement electrode 13 and the axon 3. As shown in FIG. 7(a), the axons 3 composing an axon bundle BU include axons 3A that are relatively close to the measurement electrodes 13 and axons 3B that are relatively distant from the measurement electrodes 13. Therefore, as shown in FIG. 7(b), a measurement signal corresponding to any axon 3A that is close to a measurement electrode 13 has a relatively large potential; on the other hand, as shown in FIG. 7(c), a measurement signal corresponding to any axon 3B that is distant from a measurement electrode 13 has a relatively small potential. Thus, a measurement signal that is obtained through capacitance measurement is considerably affected by changes in potential that are commensurate with the distance between a measurement electrode 13 and an axon 3.
(3) As shown in FIG. 8, a measurement signal is susceptible to a phenomenon where its baseline wobbles up and down (called a “baseline drift”). This baseline drift is another factor that may hinder evaluation of a neurotransmission signal.

### [Evaluation Apparatus and Evaluation Method]

With reference to FIG. 9, an evaluation apparatus 100 according to an embodiment of the present disclosure will be described. FIG. 9 is a diagram showing the overall configuration of a neurotransmission signal measurement system that includes the evaluation apparatus 100 (which hereinafter will be simply referred to as the “measurement system”) 200.

As shown in FIG. 9, the measurement system 200 includes the incubator 10, a data acquisition apparatus 20, and the evaluation apparatus 100.

As has already been described, the incubator 10 includes the incubation space 11, the measurement electrodes 13, and the like.

The data acquisition apparatus 20 is connected to the incubator 10, and acquires measurement data containing signal waveforms that are measured by the measurement electrodes 13 of the incubator 10. Although not shown herein, the data acquisition apparatus 20 includes a transmission section to transmit the measurement data to the evaluation apparatus 100 and the like, for example.

The evaluation apparatus 100 may be a calculation apparatus that receives the measurement data from the data acquisition apparatus 20, and performs various calculations. The evaluation apparatus 100 may be a personal computer, for example. Alternatively, the evaluation apparatus 100 may be a dedicated apparatus that functions as an assistance tool to assist in the evaluation of a neurotransmission signal.

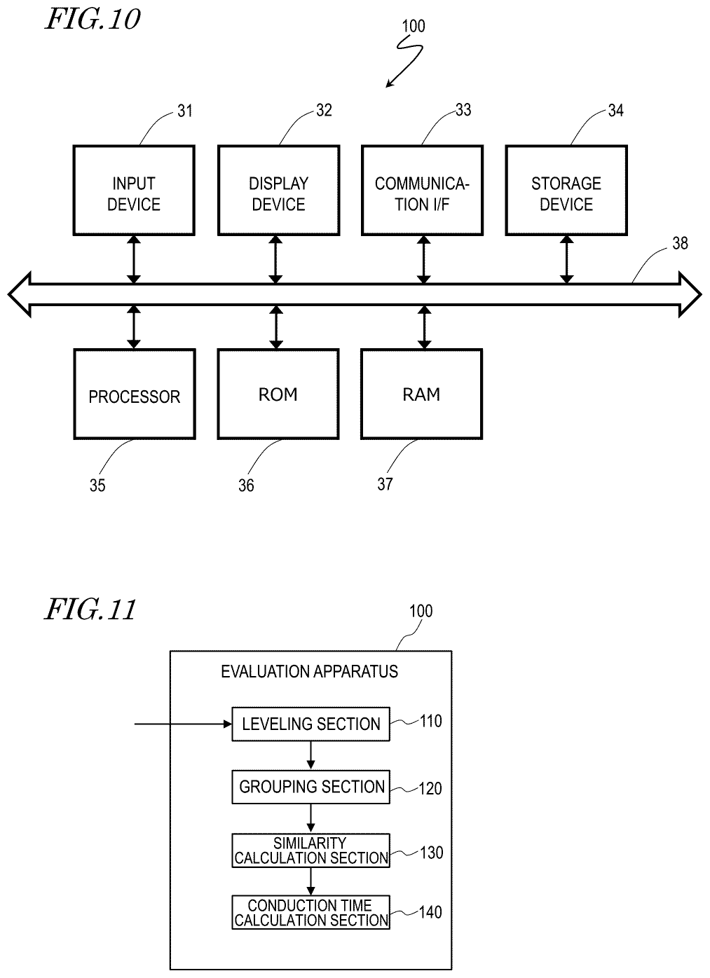

FIG. 10 is a block diagram showing an example hardware configuration for the evaluation apparatus 100. The evaluation apparatus 100 includes an input device 31, a display device 32, a communications I/F 33, a storage device 34, a processor 35, a ROM (Read Only Memory) 36, and a RAM (Random Access Memory) 37. These component elements are connected so as to be capable of communication with one another via a bus 38.

The input device 31 is a device for converting instructions from a user into data for input to a computer. The input device 31 may be a keyboard, a mouse, a touchscreen panel, or a microphone, for example.

The display device 32 may be a liquid crystal display or an organic EL display, for example. The display device 32 may display results of neurotransmission signal measurement and the like.

The communications I/F 33 is an interface for performing data communication between the evaluation apparatus 100 and the outside, and its form and protocol are not limited. For example, the communications I/F 33 is able to perform wired communication based on USB, IEEE1394 (registered trademark), or Ethernet (registered trademark), etc. The communications I/F 33 may be able to perform wireless communication under the Bluetooth (registered trademark) standards and/or Wi-Fi (registered trademark) standards. These standards all include wireless communication standards utilizing frequencies in the 2.4 GHz band.

The storage device 34 is a magnetic storage device, an optical storage device, or a combination thereof, for example. Examples of optical storage devices include optical disc drives, magneto-optical disc (MD) drives, and the like. Examples of magnetic storage devices include hard disk drives (HDD), floppy disk (FD) drives, and magnetic tape recorders.

The processor 35 is a semiconductor integrated circuit, also referred to as a central processing unit (CPU) or a microprocessor. The processor 35 consecutively executes a computer program that is stored in a ROM 160 to achieve desired processes. The processor 35 is to be broadly interpreted as a term that encompasses an FPGA (Field Programmable Gate Array), an ASIC (Application Specific Integrated Circuit) or an ASSP (Application Specific Standard Product) on which a CPU is mounted.

The ROM 36 may be a writable memory (e.g., a PROM), a rewritable memory (e.g., a flash memory), or a read-only memory, for example. The ROM 36 stores a program for controlling the operation of the processor. The ROM 36 does not need to be a single storage medium, but may be an aggregation of multiple storage media. A part of such an aggregate storage may be a removable memory.

The RAM 37 provides a work area for a control program stored in the ROM 36 to be laid out once at boot time. The RAM 37 does not need to be a single storage medium, but may be an aggregation of multiple storage media.

FIG. 11 is a functional block diagram showing the evaluation apparatus 100 in functional blocks. As shown in FIG. 11, the evaluation apparatus 100 includes a leveling section 110, a grouping section 120, a similarity calculation section 130, and a conduction time calculation section 140.

The evaluation apparatus 100 evaluates a neurotransmission signal that is conducted through the axon bundle BU from a signal waveform that is acquired by using each of the plurality of measurement electrodes 13. As will be seen from what has been described, the plurality of measurement electrodes 13 are disposed at respectively different positions relative to the axon bundle BU.

The leveling section 110 performs leveling for the acquired signal waveform. “Leveling” is a process of estimating a baseline of a signal waveform and subtracting the estimated baseline from the signal waveform.

The grouping section 120 groups a plurality of peaks that are contained in the signal waveform after having undergone the leveling into a plurality of groups. As used herein, the “plurality of groups” respectively correspond to one of the axons 3 included in the axon bundle BU. In other words, grouping means identifying two or more peaks that pertain to the same axon 3.

After the grouping by the grouping section 120, the similarity calculation section 130 calculates a similarity between: a signal waveform (hereinafter referred to as the “first signal waveform”) acquired by a certain measurement electrode 13 (hereinafter denoted as the “first measurement electrode 13A”) among the plurality of measurement electrodes 13; and a signal waveform (hereinafter referred to as the “second signal waveform”) acquired by another measurement electrode 13 (hereinafter referred to as the “second measurement electrode 13B”).

Based on the calculated similarity, the conduction time calculation section 140 calculates a signal conduction time between the first measurement electrode 13A and the second measurement electrode 13B.

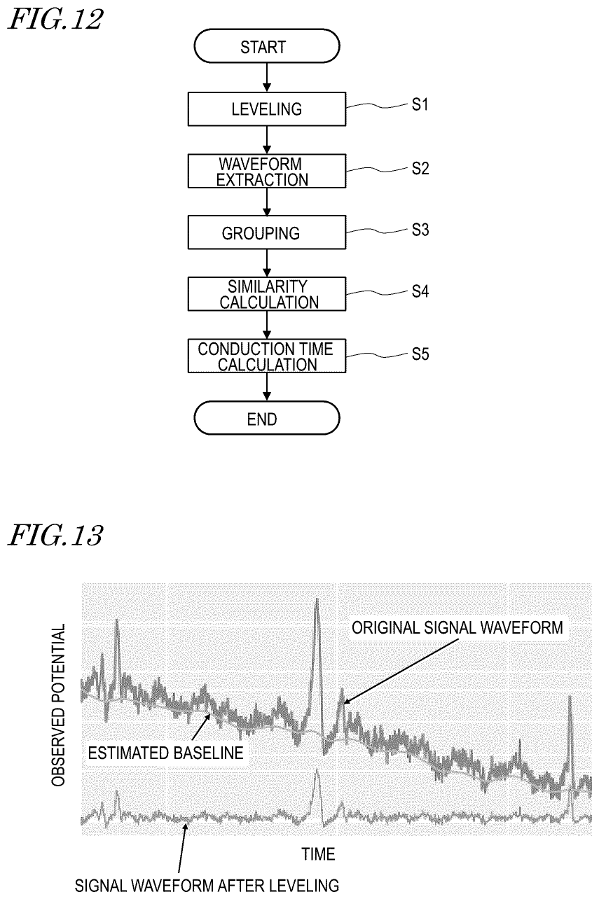

With the aforementioned configuration, the evaluation apparatus 100 according to an embodiment of the present disclosure is able to suitably evaluate a neurotransmission signal that is conducted through the axon bundle BU of the 3D organoid 5. Hereinafter, with reference to FIG. 12, a specific example of a processing procedure (algorithm) by the evaluation apparatus 100 will be described. A computer program including instructions that describe the algorithm may be distributed via the Internet, or marketed as packaged software, for example. FIG. 12 is a flowchart showing an example processing procedure.

First, the leveling section 110 performs leveling for the acquired signal waveform (step S1). For example, baseline estimation can be suitably performed by using an asymmetric least squares method.

An asymmetric least squares method is a technique that is used in spectroscopic studies or the like, as disclosed in Non-Patent Document 1, for example. The entire enclosure of Non-Patent Document 1 is incorporated herein by reference. In an asymmetric least squares method, an evaluation function is expressed by formula (1) below.

$$\begin{matrix}[ math . 1 ] & \\ S = {\sum }_{i} {{ω}_{i} ( {y}_{i} - {z}_{i} )}^{2} + λ {\sum }_{i} {( {Δ}^{2} {z}_{i} )}^{2} & ( 1 )\end{matrix}$$

In formula (1), yᵢ is a measured value, and zᵢ is an estimated value. The first term in this evaluation function is a term that indicates a degree of fit of the estimated value, whereas the second term is a penalty term to adjust smoothness (degree of extension). wᵢ is a weight, such that: the weight wᵢ is p (where p is a parameter) when the difference between the measured value yᵢ and the estimated value zᵢ is positive (i.e., when yᵢ−zᵢ>0); and the weight wᵢ is 1-p when the difference between the measured value yᵢ and the estimated value zᵢ is negative (i.e., when yᵢ-zᵢ<0). λ is a parameter for adjusting the balance between these two terms.

As has already been described, a measurement signal is susceptible to a phenomenon where its baseline wobbles up and down (baseline drift), and this baseline drift is a factor that hinders measurement of a neurotransmission signal. Merely applying a filter to such a measurement signal, or deriving a moving average, will distort the characteristics of the signal. On the other hand, by using an asymmetric least squares method to estimate a baseline and subtracting it from the original signal waveform, for example, it is possible to suitably perform waveform extraction without much distorting the characteristics of the original signal waveform.

An example of subtracting an estimated baseline from the actual signal waveform is illustrated in FIG. 13. FIG. 13 shows the original signal waveform and the estimated baseline, as well as a signal waveform after leveling. It can be seen from FIG. 13 that the influences of the baseline drift are eliminated.

Then, from the signal waveform after leveling, a desired portion (a portion to be subjected to the grouping described below) is extracted (step S2).

Next, the grouping section 120 groups a plurality of peaks that are contained in the signal waveform after having undergone the leveling into a plurality of groups (step S3). The grouping may involve, with respect to a given pair of peaks among the plurality of peaks, calculating a time difference between peaks, and be performed based on the calculated time difference and the peak heights, for example. Since the axon 3 transmits information based on the transmission interval of signals and the intensity thereof, it is possible to perform grouping (i.e., identifying two or more peaks that pertain to the same axon 3) based on the time difference between peaks and the peak heights.

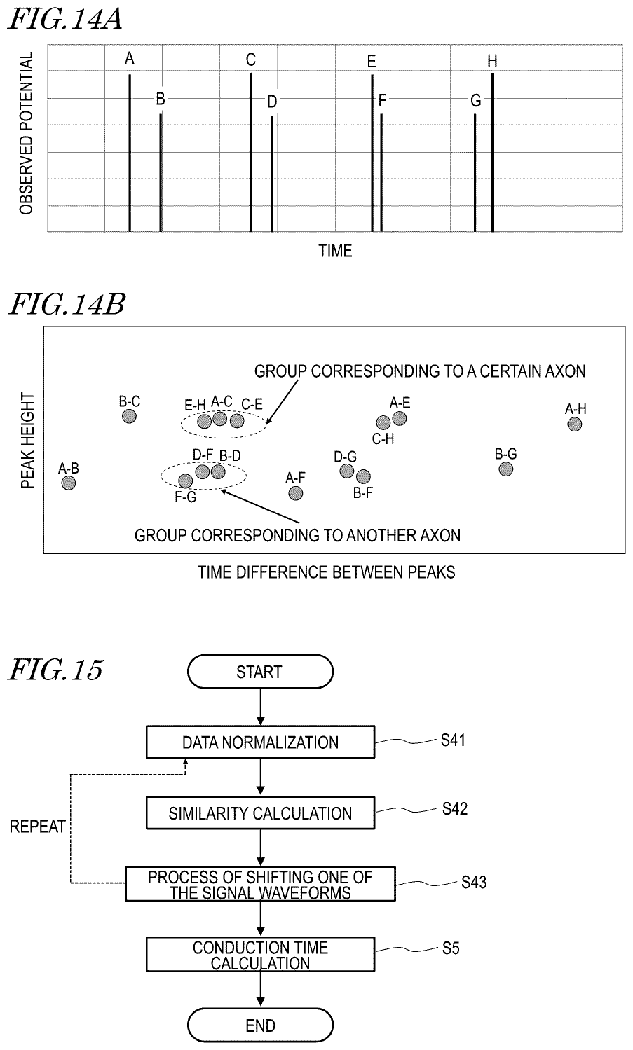

For example, consider a case where eight peaks A to H as shown in FIG. 14A are grouped. With respect to a given pair of peaks (hereinafter simply referred to as a “pair”) among these eight peaks, a time difference between the peaks is calculated. FIG. 14B is a graph where each pair is denoted in the order of the earlier peak and the later peak (for example, a pair of peak A and peak B is denoted as “A-B”), and each pair is plotted with the time difference between the peaks reading on the horizontal axis and the peak height of the later peak reading on the vertical axis.

As shown in FIG. 14B, peaks A, C, E and H, which are contained in mutually proximate (i.e., essentially at the same position) pairs “A-C”, “C-E” and “E-H”, may be determined as corresponding to the same axon. Peaks B, D, F and G, which are contained in mutually proximate (i.e., essentially at the same position) pairs “B-D”, “D-F” and “F-G”, may be determined as corresponding to the same axon.

The grouping by the grouping section 120 may be semi-automated (or fully-automated) by using machine learning. As a technique of machine learning, clustering can be used, for example. An example of clustering may be the k-means method.

Then, the similarity calculation section 130 calculate a similarity between: a signal waveform (first signal waveform) acquired by a certain measurement electrode 13 (first measurement electrode 13A); and a signal waveform (second signal waveform) acquired by another measurement electrode 13 (second measurement electrode 13B) (step S4).

Thereafter, based on the calculated similarity, the conduction time calculation section 140 calculates a signal conduction time between the first measurement electrode 13A and the second measurement electrode 13B (step S5).

The similarity calculation step S4 and the conduction time calculation step S5 can be suitably performed as follows, for example. In the similarity calculation step S4, for vicinities of all peaks in a first signal waveform that belong to a certain group (i.e., pertaining to the same axon 3), inner product values with a second signal waveform obtained by being shifted by a predetermined shift amount in a time axis direction are calculated and added up, the resultant value thereof being determined as the similarity. Then, the similarity calculation step S4 is performed a plurality of times while varying the shift amount; and, at the conduction time calculation step S5, a shift amount that maximizes the similarity is determined as a signal conduction time. As a result, similar aspects can be emphasized while suppressing noise, whereby similarity calculation and conduction time calculation can be suitably performed. Hereinafter, with reference to FIG. 15 to FIG. 20, this technique will be described more specifically. FIG. 15 is a flowchart of a specific technique of similarity calculation and conduction time calculation.

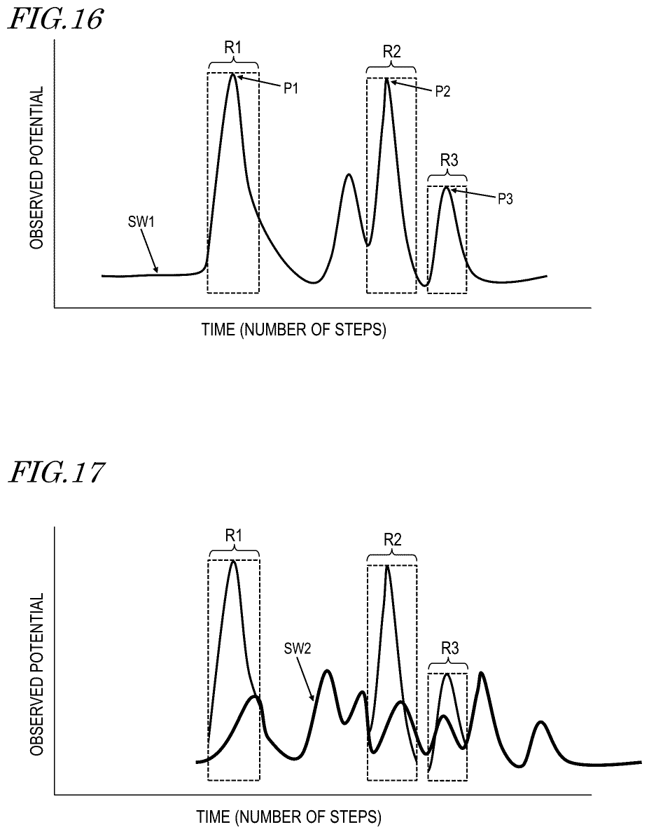

First, from a first signal waveform, vicinities of all peaks (hereinafter referred to as “peak vicinities”) that are determined as belonging to the same group by grouping are extracted. A “peak vicinity” means a predetermined interval that includes a peak (e.g., substantially centered around the peak). Although the length of the interval is not particularly limited, if one observation step of signal potential takes 0.05 ms, it may be 300 steps, for example. FIG. 16 shows an example of peak vicinities that may be extracted from a first signal waveform. In the example shown in FIG. 16, from a first signal waveform SW1, peak vicinities R1, R2 and R3 are extracted with respect to peaks P1, P2 and P3 belonging to a certain group. At this time, as shown in FIG. 17, the same intervals (intervals corresponding to the peak vicinities R1, R2 and R3) of a second signal waveform SW2 are also extracted. Also, at this time, a pair consisting of each of the peak vicinities R1, R2 and R3 of the first signal waveform SW1 and a corresponding extraction interval in the second signal waveform SW2 is subjected to normalization for a maximum value of 1 (normalized around zero while maintaining large-small relationships) (step S41 in FIG. 15).

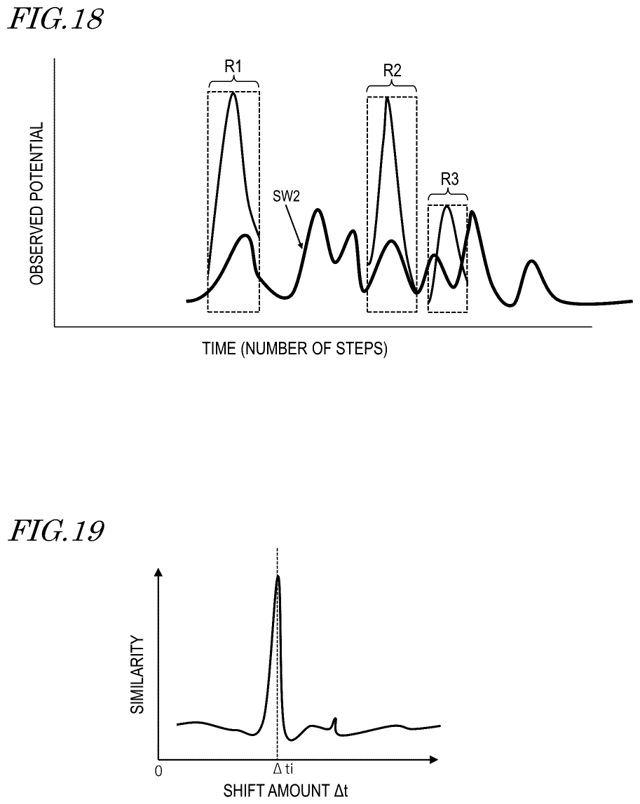

Next, inner product values of the respective pairs are calculated and added up, and the resultant value is determined as a similarity (step S42). Then, as shown in FIG. 18, a process of shifting the second signal waveform SW2 in a time axis direction (e.g., in a negative direction as in here) by a predetermined shift amount Δt is performed (step S43). Thereafter, normalization, similarity calculation, and a process of shifting the second signal waveform SW2 are repeated a predetermined number of times.

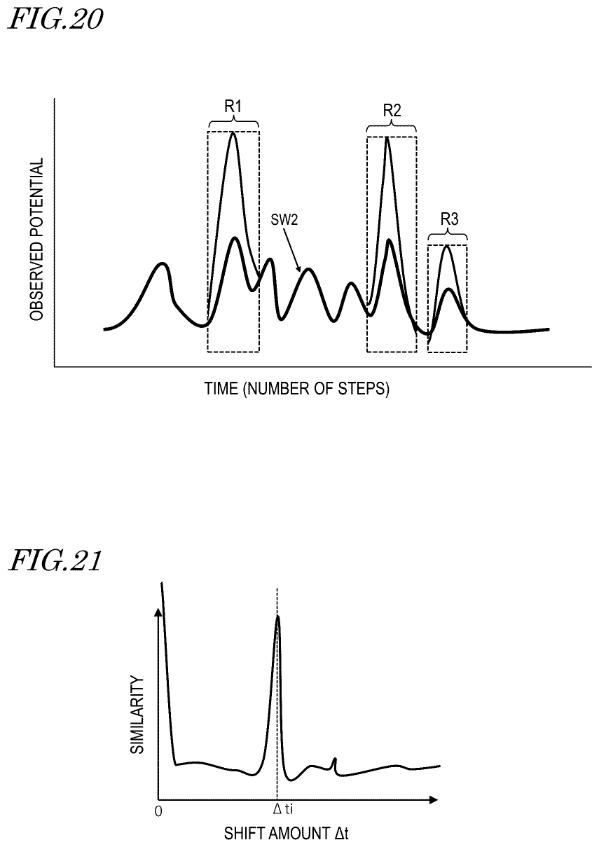

Thereafter, a shift amount Δti that maximizes the similarity is calculated as a signal conduction time (step S5). FIG. 19 is a graph to visualize a relationship between the shift amount Δt and the similarity where the shift amount Δt reads on the horizontal axis and the similarity reads on the vertical axis; and FIG. 20 shows a state where the second signal waveform SW2 has been shifted by the shift amount Δti that maximizes the similarity. It can be seen from FIG. 19 that the similarity becomes maximum at a certain shift amount Δti. The shift amount Δti that maximizes the similarity can be regarded as a delay time in the waveform between the first measurement electrode 13A and the second measurement electrode 13B, i.e., signal conduction time.

Note that, as shown in FIG. 21, due to noise components residing on the entire measurement system, the calculated similarity may take a greater value in the neighborhood of shift amount Δt=0 than what is really the maximum value. For this reason, it is preferable to eliminate the neighborhood of Δt=0 from the evaluation, and the shift amount Δt is to be set to an appropriate value that is greater than 0. The shift amount Δt may be set in a range of s/vmax to s/min, which is calculated backwards from a range of vmin to vmax of signal speed that is expectable to the skilled artisan and the distance s between electrodes.

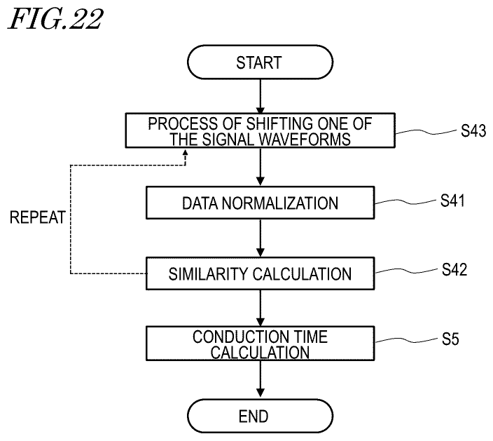

For ease of understanding, the flowchart shown in FIG. 15 illustrates an example where the similarity calculation is first performed in a state of shift amount Δt=0. However, an initially-calculated similarity (i.e., the similarity in the case of Δt=0) may be eliminated at the time of conduction time calculation; or, as shown in FIG. 22, the second signal waveform SW2 may already be shifted in the beginning. In the example shown in FIG. 22, a process of shifting the second signal waveform SW2 in a time axis direction (e.g., in a negative direction as in here) by a predetermined shift amount Δt is first performed (step S43), and then data normalization (step S41) and similarity calculation (step S42) are performed in order.

Note that the aforementioned technique may be regarded as an application of Code Division Multiple Access (CDMA). By applying CDMA, similar aspects can be emphasized while suppressing noise, whereby similarity calculation and conduction time calculation between waveforms can be suitably performed.

Thus, the evaluation apparatus 100 according to an embodiment of the present disclosure includes the leveling section 110, the grouping section 120, the similarity calculation section 130, and the conduction time calculation section 140, thereby being able to suitably evaluate a neurotransmission signal that is conducted through the axon bundle BU.

As has been illustrated, by using an asymmetric least squares method to perform a baseline estimation at the time of leveling, waveform extraction can be suitably performed without much distorting the characteristics of the original signal waveform.

With respect to a given pair of peaks among a plurality of peaks, a time difference between peaks is calculated, and the calculated time difference and peak heights are utilized to suitably perform grouping (identifying two or more peaks that pertain to the same axon 3).

Furthermore, by using the aforementioned technique which is an application of CDMA, similar aspects can be emphasized while suppressing noise, whereby similarity calculation and conduction time calculation can be suitably performed.

### INDUSTRIAL APPLICABILITY

According to an embodiment of the present disclosure, an evaluation method and an evaluation apparatus which can evaluate a neurotransmission signal that is conducted through an axon bundle can be provided.

### REFERENCE SIGNS LIST

1 nerve cell
2 cell body
2a nucleus
3 axon
3a axon terminal
5 3D organoid
10 incubator (microfluidic device)
11 incubation space
11a first chamber
11b second chamber
11c channel
12 substrate
13 measurement electrode
14 upper plate
culture fluid
16 cultivation substrate
17 coating layer
20 data acquisition apparatus
100 evaluation apparatus
200 measurement system
110 leveling section
120 grouping section
130 similarity calculation section
140 conduction time calculation section
BU axon bundle
CL mass of cell bodies

1. An evaluation method for evaluating a neurotransmission signal that is conducted through an axon bundle, which is a bundle of axons of a plurality of nerve cells, an evaluation being based on a signal waveform that is acquired by using each of a plurality of measurement electrodes disposed at respectively different positions relative to the axon bundle, the evaluation method comprising:
performing a leveling that estimates a baseline of the signal waveform and subtracts an estimated baseline from the signal waveform;
grouping a plurality of peaks that is contained in the signal waveform after the leveling into a plurality of groups each corresponding to one of the axons included in the axon bundle;
calculating a similarity between a first signal waveform acquired by a first measurement electrode among the plurality of measurement electrodes and a second signal waveform acquired by a second measurement electrode, which is distinct from the first measurement electrode, after the grouping; and
calculating a signal conduction time between the first measurement electrode and the second measurement electrode, based on a calculated similarity.

2. The evaluation method of claim 1, wherein in the leveling, the baseline is estimated by using an asymmetric least squares method.

3. The evaluation method of claim 1, wherein in the grouping, with respect to a given pair of peaks among the plurality of peaks, a time difference between the plurality of peaks is calculated, and the grouping is performed, based on a calculated time difference and peak heights of the given pair of peaks.

4. The evaluation method of claim 1, wherein in the calculating of the similarity, for vicinities of all peaks in the first signal waveform that belong to a certain group, inner product values with a second signal waveform obtained by being shifted by a predetermined shift amount in a time axis direction are calculated and added up, and a resultant value thereof is determined as the similarity.

5. The evaluation method of claim 4, wherein
the calculating of the similarity is performed a plurality of times while varying the predetermined shift amount; and,
in the calculating of the signal conduction time, a shift amount that maximizes the similarity is determined as the signal conduction time.

6. An evaluation apparatus for evaluating a neurotransmission signal that is conducted through an axon bundle, which is a bundle of axons of a plurality of nerve cells, an evaluation being based on a signal waveform that is acquired by using each of a plurality of measurement electrodes disposed at respectively different positions relative to the axon bundle, the evaluation apparatus comprising:
a leveling section to perform a leveling that estimates a baseline of the signal waveform and subtracts an estimated baseline from the signal waveform;
a grouping section to group a plurality of peaks that is contained in the signal waveform after the leveling into a plurality of groups each corresponding to one of the axons included in the axon bundle;
a similarity calculation section to calculate a similarity between a first signal waveform acquired by a first measurement electrode among the plurality of measurement electrodes and a second signal waveform acquired by a second measurement electrode, which is distinct from the first measurement electrode, after the grouping by the grouping section; and
a conduction time calculation section to calculate a signal conduction time between the first measurement electrode and the second measurement electrode, based on a calculated similarity.

7. The evaluation apparatus of claim 6, wherein the leveling section estimates the baseline by using an asymmetric least squares method.

8. The evaluation apparatus of claim 6, wherein with respect to a given pair of peaks among the plurality of peaks, the grouping section calculates a time difference between the plurality of peaks, and performs grouping, based on a calculated time difference and peak heights of the given pair of peaks.

9. The evaluation apparatus of claim 6, wherein for vicinities of all peaks in the first signal waveform that belong to a certain group, the similarity calculation section calculates and adds up inner product values with a second signal waveform obtained by being shifted by a predetermined shift amount in a time axis direction, and a resultant value thereof is determined as the similarity.

10. The evaluation apparatus of claim 9, wherein
the similarity calculation section calculates the similarity a plurality of times while varying the shift amount; and
the conduction time calculation section determines a shift amount that maximizes the similarity as the signal conduction time.

11. A computer program for evaluating a neurotransmission signal that is conducted through an axon bundle, which is a bundle of axons of a plurality of nerve cells, an evaluation being based on a signal waveform that is acquired by using each of a plurality of measurement electrodes disposed at respectively different positions relative to the axon bundle, the computer program including instructions, which when executed by one or more processors, cause the one or more processors to:
a leveling that estimates a baseline of the signal waveform and subtracts an estimated baseline from the signal waveform;
grouping a plurality of peaks that is contained in the signal waveform after the leveling into a plurality of groups each corresponding to one of the axons included in the axon bundle;
calculating a similarity between a first signal waveform acquired by a first measurement electrode among the plurality of measurement electrodes and a second signal waveform acquired by a second measurement electrode, which is distinct from the first measurement electrode, after the grouping; and
calculating a signal conduction time between the first measurement electrode and the second measurement electrode, based on a calculated similarity.
